(19) United States
(12) Patent Application Publication
(10) Pub. No.: US 2020/0002668 A1
(43) Pub. Date: Jan. 2, 2020

(54) CONTROL OF CELL GROWTH AND AGGREGATE SIZE IN BIOREACTORS

(71) Applicant: PBS Biotech, Inc., Camarillo, CA (US)

(72) Inventors: Chanyong Brian LEE, Newbury Park, CA (US); Sunghoon JUNG, Camarillo, CA (US); Yasunori Hashimura, Woodland Hills, CA (US)

(21) Appl. No.: 16/460,021

(22) Filed: Jul. 2, 2019

Related U.S. Application Data
(60) Provisional application No. 62/693,238, filed on Jul. 2, 2018.

(51) Int. Cl. C12N 5/00

(52) U.S. Cl. CPC C12N 5/0075; C12N 2531/00

(57) ABSTRACT

Methods of repeated aggregate dissociation and reformation of pluripotent stem cells (PSCs) within the same bioreactor until a desired final cell number is achieved. A preferred step-wise process for controlled growth of PSCs and aggregate size using periodic dissociation with a dissociation medium which contains either proteolytic enzymes or chemical reagents, mechanical agitation, or a combination of these methods.

### RELATED APPLICATION INFORMATION

This application claims priority from Provisional Patent Application No. 62/693,238, filed Jul. 2, 2018, titled CONTROL OF CELL GROWTH AND AGGREGATE SIZE IN BIOREACTORS, which is expressly incorporated herein by reference.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### FIELD OF THE INVENTION

Systems and methods for controlling the size of anchorage-dependent cell aggregates or cells and microcarriers clumped together in bioreactors, in order to maximize total cell growth within a given bioreactor.

### BACKGROUND

Pluripotent stem cells (PSCs) can be derived from human embryos or by inducing pluripotency in adult somatic cells. The distinguishing characteristic of PSCs is their ability to differentiate into virtually any cell type in the human body, which makes them a promising cell therapy tool to potentially treat a wide variety of different disease indications. Furthermore, PSCs can grow (proliferate) almost indefinitely in culture, which is critical to meet dosage needs that can range from millions to even billions of cells per person. A unique requirement of PSC manufacturing is that, after completing a cell expansion phase to maximize total cell growth, a differentiation phase is then needed to direct the cells to turn into a target cell type. For instance, FIG. 1 shows how a PSC such as a human embryonic stem cell (ESC) or induced pluripotent stem cell (iPSC) can be directed to become a target cell type through multiple, sequential directed differentiation steps using specific growth factors (GF) for each step.

Attempting to produce a huge magnitude of cells at commercial scale using traditional 2D manufacturing platforms would be extremely cost prohibitive and thus infeasible. Instead, 3D suspension culture in a bioreactor represents the best option for scaled-up expansion and differentiation of PSCs.

Currently, the most common method for seed train culturing of PSCs involves initial seeding from a frozen vial and expansion in a planar plate as a 2D monolayer culture. The monolayer cells are then harvested and dissociated into single cells or small cell clusters, which may then be seeded into a 3D culture vessel, such as an instrumented bioreactor, for scale up as a suspension culture in a controlled environment. The single cells or small-size clusters will then automatically form spherical cell aggregates that will continue to grow in size, through the proliferation of cells within the aggregates as well as further attachment of free-floating single cells or small clumps. Within a bioreactor, both the total cell number and size of cell aggregates will increase along with cultivation time under appropriate culture conditions as long as the required nutrients or key growth factors are continuously provided in the medium, metabolites do not accumulate to harmful levels, and the medium components can diffuse to the cells located in the center of aggregates.

Due to a surge in demand for PSCs for research and other purposes, there is a need for process techniques to consistently and reliably produce large quantities of high quality PSCs. Despite cell aggregation being a naturally occurring and desirable outcome for PSC cell growth, there are potential downsides if the cell aggregates become too large in size. Similarly, cells that are attached to the surface of microcarriers may stick together creating microcarrier clumps, which may be undesirable.

### SUMMARY OF THE INVENTION

The present application discloses systems and methods for controlling the size and growth of cells aggregates or cells and microcarriers clumped together in bioreactors, in order to achieve a desired cell density or total cell amount within a given bioreactor.

### DESCRIPTION OF THE DRAWINGS

FIG. 1 shows a multi-stage directed differentiation process for generation of a target differentiated cell from an initial PSC in vitro;

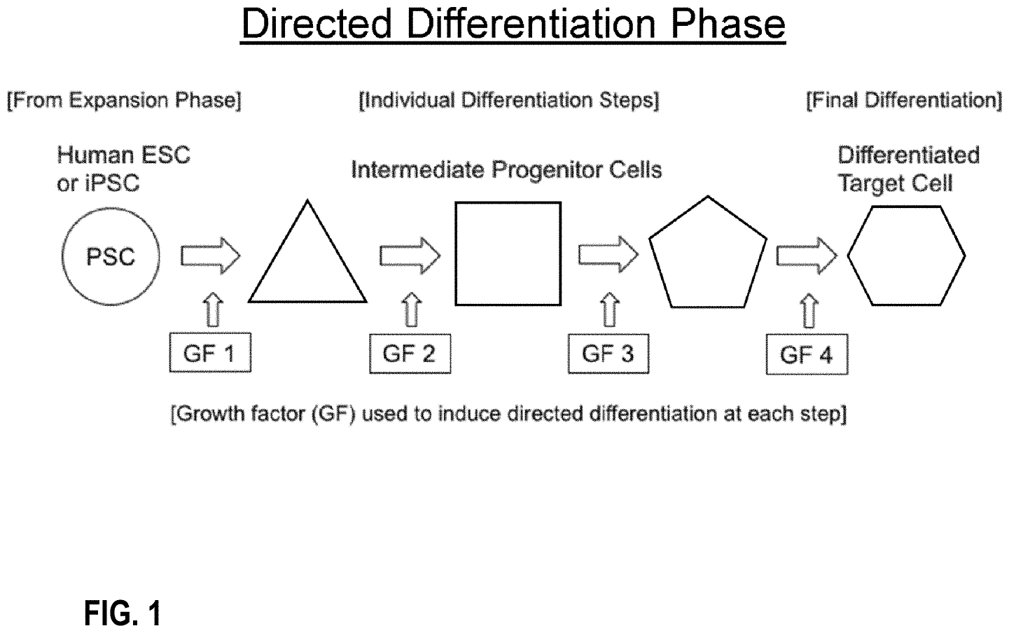

FIG. 2 is a graph that indicates a preferred step-wise process for controlled growth of PSCs and aggregate size using periodic dissociation.

### DETAILED DESCRIPTION

The present application provides systems and methods for controlling the size and growth of cells aggregates or cells and microcarriers clumped together in bioreactors. Although the techniques described herein may be most useful for producing large quantities of high quality PSCs, they may also be utilized to improve the production efficiency of other anchorage-dependent cell types such as mesenchymal stem cells (MSCs), human primary cells, or cells grown on microcarriers. In the case of cells grown on the surface of microcarriers, the formation of undesirable clumps of cells and microcarriers stuck together may occur. For instance, the methods can be used to separate cell-and-microcarrier clumps into much smaller clumps of acceptable size, back into individual microcarriers with cells still attached to their surfaces, or even to completely remove cells from microcarriers. Therefore, the present application should not be considered limited to the production of PSCs.

Additionally, various 3D culture vessel bioreactors may be used to produce cells such as PSCs. In a preferred embodiment, a vertical-wheel bioreactor is used for its relatively low shear rate and thus low damage to PSCs. However, other bioreactor configurations such as those with horizontal stirring paddles or impellers, or other methods of agitation, may be used.

Potential issues with medium diffusion may occur if cell aggregates become too large (e.g., diameters greater than 500 μm). During cell expansion, nutrients in the growth medium may no longer be able to diffuse to the cells in the center of large aggregates, leading to cell damage or even necrosis. Similarly during differentiation, the soluble differentiation-inducing factors that would direct the formation of target cells may also be unable to reach the cells in the center of large aggregates. Furthermore, the inability of certain medium components (such as regulatory factor including cytokines) to reach the center of cell aggregates could result in a non-homogeneous population of PSCs during the expansion phase, or undesired differentiated cells during the differentiation phase. Finally, damaging shear effects may become more pronounced on outer cells as the diameter of cell aggregates becomes much larger than eddy sizes. Therefore, overly large cell aggregates can negatively impact the cell quality, differentiation efficiency, and final yield of PSCs during 3D scale-up. For each type of PSC, there should exist an ideal aggregate size that allows nutrients and required medium components to reach its center while maximizing cell expansion. Similarly, for each type of target differentiated PSC, there should exist an ideal aggregate size that allows for differentiation-inducing factors to reach its center to allow the most efficient directed differentiation with high yield. Therefore, a desired total cell yield cannot be achieved in a single bioreactor simply by increasing cultivation time and providing cells with a sufficient amount of medium until the vessel reaches full capacity, due to the aforementioned disadvantages associated with overly large aggregates. Trying to maximize the quantity of cells in a single bioreactor by letting them grow continuously leads to large aggregates, which negatively affects the quality of the cells for continued expansion and eventual differentiation.

Historically, expansion of PSCs is achieved by initially growing cells in a small bioreactor until the cell aggregates reach a certain size, dissociating the aggregates, and then transferring the batch of cells into a larger-size bioreactor with fresh medium. Dissociating the cell aggregates back into single cells or small-size clusters can be accomplished by using either a dissociation medium containing proteolytic enzymes or chemical reagents, mechanical agitation, or a combination of these methods. In the present application the term “dissociation” encompasses all of these methods.

In any event, conventional expansion of PSCs occurs by dissociating cell aggregates once they reach a predetermined size and then passaging the resulting single cells or small clusters into a larger volume bioreactor with fresh medium to continue expansion. When the dissociated single cells or small clusters are passaged, any dissociation medium that was used would need to be removed or inactivated by the use of a counter-acting reagent to allow the cells to reform into aggregates and grow in the new bioreactor. This process of dissociation and passaging into progressively larger bioreactors continues until a final cell yield is achieved, completing the expansion phase. However, avoiding cell damage or necrosis from overly large aggregates typically requires dissociation and passaging to occur before a bioreactor's full volumetric capacity is utilized, which is an inefficient use of equipment, labor, and expensive PSC culture media. These high operational costs become exponentially more pronounced during scale-up to commercial manufacturing. Also, the total cell yield achievable in the expansion phase is affected by how many sequentially larger bioreactors are available for passaging, and would be even more limited if those bioreactors were used at less than full capacity. Therefore, a more efficient process that maximizes a given-sized bioreactor's capacity while maintaining the optimal size of cell aggregates will significantly improve the large-scale commercial manufacturing of PSCs.

Controlling and limiting the size to which PSC aggregates grow during the expansion phase in a bioreactor can greatly increase both the efficiency and final yield of differentiated target cells. As opposed to methods that involve passaging into sequentially larger bioreactors, this can be achieved in single bioreactor by periodically dissociating the large aggregates into single cells or smaller cell clusters, and then allowing them to reform into aggregates that will again grow in size while increasing the total cell yield.

Additionally, cells at the end of each dissociation step will be at higher volumetric concentration compared to the preceding dissociation step as a result of cell proliferation, assuming that more liquid medium was not added. Consequently, the frequency of collisions between single cells or small clusters and cell aggregates is expected to be higher if the same hydrodynamic shear stress is maintained, which can accelerate the formation of overly large cell aggregates. Gradually increasing the bioreactor's impeller agitation rate at each subsequent cell re-aggregation step may therefore be necessary to maintain a constant initial cell aggregate size at each re-aggregation step.

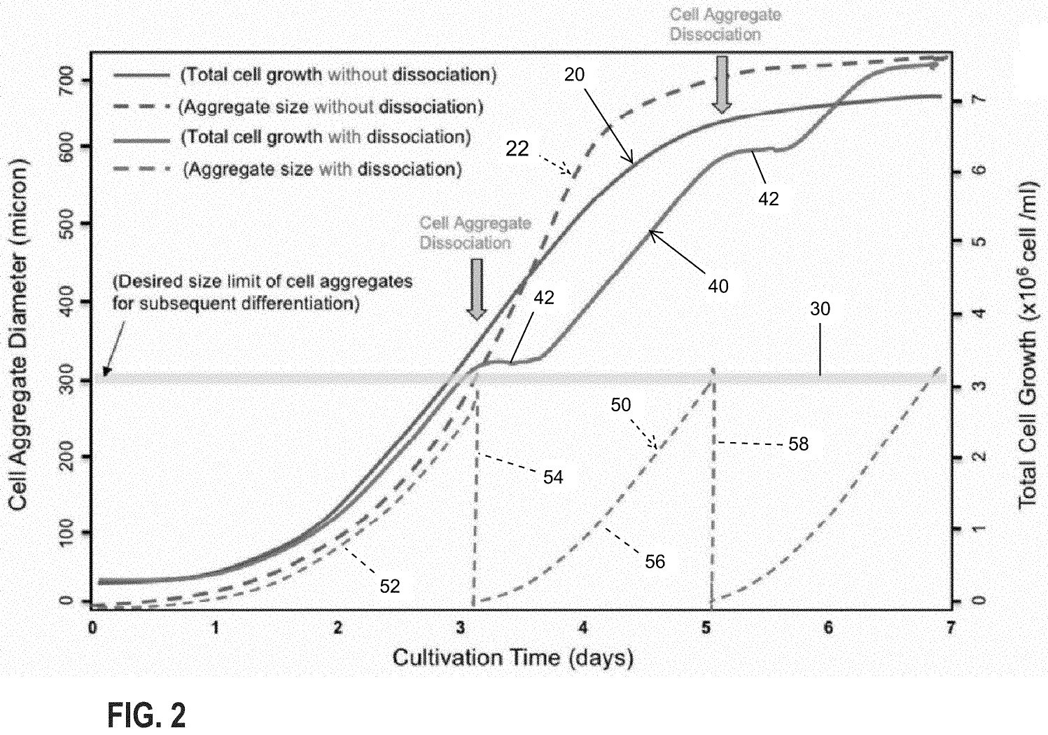

In accordance with the present application, a process of aggregate dissociation and reformation within the same bioreactor can be repeated until the desired final cell number is achieved. For instance, FIG. 2 is a graph that indicates a preferred step-wise process for controlled growth of PSCs and aggregate size using periodic dissociation.

Cultivation time in days is shown on the X-axis, and both cell aggregate diameter (left) and total cell growth (right) are shown on the Y-axes. The smoothly curved solid blue (dark) line 20 shows total cell growth without any dissociation, while the smoothly curved broken blue (dark) line 22 indicates aggregate size growth without any dissociation. These two smooth curves represent the typical growth of both total number of cells and aggregate size in the absence of any dissociation. Both graph lines 20, 22 increase generally in the same way, with a relatively steep initial ascent followed by a leveling off at practical maximums of both total cells and aggregate size. Although such an increase in the total number of cells is desirable, the accompanying growth of the size of the cell aggregates is not. That is, as stated above, overly large cell aggregates can negatively impact the cell quality, differentiation efficiency, and final yield of PSCs.

Horizontal line 30 indicates an exemplary desired maximum size threshold for cell aggregates, which in this graph is set at about 300 microns. The broken line 22 indicating aggregate size in the absence of dissociation rapidly exceeds the desired size threshold 30 relatively soon in the cultivation process. Although cells continue to be produced, the agglomeration into aggregates limits the quality of the yield. The threshold line 30 may differ for different batches of PSCs and different processing conditions.

To resolve this detrimental phenomenon, the present application proposes a process of periodic dissociation of the cell population to periodically break up the accumulated aggregates within the same bioreactor. In such a process, solid red (light) line 40 represents the total number of cells produced while broken red (light) line 50 indicates aggregate size growth with periodic dissociation. Solid line 40 grows generally continuously, with steps or plateaus 42 corresponding to periodic dissociation events. Broken line 50 grows initially along curve 52 to threshold line 30 at which point a dissociation event occurs in the original bioreactor, and the cell aggregate size drops precipitously at 54 to near zero (no cell aggregates).

Subsequently, further cultivation initiates cell aggregate growth along curve 56 until the next dissociation event and drop in aggregate size at 58, and so on. Periodic dissociation in this manner prevents the aggregate size from exceeding the threshold line 30 which greatly improves quality of the PSCs while continuing to grow the total number of cells (line 40). Ultimately, after 7 days in the same bioreactor, the total number of cells shown in line 40 has grown to a similar magnitude as the number of cells without dissociation (line 20), in this case around 8×10⁶ cells/ml, but the aggregate size shown by line 50 is at or below the threshold line 30.

The benefits of this process are that it controls the size of aggregates, maximizes aggregate number and total cell yield, maintains a healthy and homogenous cell population throughout the expansion and differentiation phases, and utilizes the full capacity of a given size bioreactor before transferring to a larger size bioreactor as needed. This can improve process efficiency and minimize the different sizes and total number of bioreactors necessary for commercial manufacturing of PSCs, reducing the cost of goods significantly. These benefits can also apply to other anchorage-dependent cell types that can form aggregates or are grown on microcarriers, which can then form clumps of cells and microcarriers.

To induce dissociation of the cell aggregates, new medium containing specific dissociation reagents or enzymes can be added through a medium exchange process, and in combination with mechanical agitation as needed. This dissociation activity needs to be quenched once all the aggregates are dissociated into small clumps or single cells. This can be accomplished by adding another reagent such as serum or platelet lysate to counteract dissociation reagents and then diluting this activity further with additional growth medium, or by utilizing an in-situ cell retention device or external cell separation device to perform complete medium exchange to replace the dissociation medium with growth medium. The steps of dissociation, quenching, and medium exchange can be repeated periodically throughout the expansion phase to achieve optimal aggregate size and the desired total cell number, at which point a final medium exchange step using a differentiation medium can be performed to move onto the differentiation phase.

A complete medium exchange can be quickly and effectively performed in bioreactors using an external cell separation device. For example, after a certain time period of cell growth as aggregates in a bioreactor and before the cell aggregates become too large (resulting in cell damage or necrosis at the center of aggregates), agitation would be stopped to allow the aggregates to sink to the bottom of the bioreactor through gravity. The majority of growth medium can then be removed from the supernatant and replaced by dissociation medium (i.e. fresh growth medium containing dissociation reagents). Agitation could then be restarted to promote the dissociation of aggregates into small clumps or single cells, as well as to resuspend them. The dissociation medium containing small aggregates/clumps or single cells would then be pumped to a cell separation device outside the bioreactor, where it can be concentrated and washed with fresh growth medium. The growth medium containing concentrated small aggregates/clumps or single cells could then be transferred back into the original bioreactor which has been preconditioned (in parameters such as temperature, pH, DO, etc.) with the same growth medium. In case that the preconditioning of the original bioreactor cannot be completed during the concentration and wash steps in the external device, a same size, second bioreactor can be prepared instead. Once returned to the bioreactor, the small aggregates/clumps or single cells will reform aggregates while continuing to expand the total cell number. The medium exchange process, from growth medium to dissociation medium and back again, can be repeated multiple times until the desired total cell number is achieved. By transferring as single cells or small clusters instead of large aggregates to the external device, the potential damage of cell aggregates from mechanical shear stress by peristaltic pumps is also greatly reduced.

Certain anchorage-dependent cells grow ideally as 2D monolayers on the surface of microcarriers while suspended in a bioreactor. These cells may then stick to each other through cell-to-cell attachment and form undesirable clumps of cells and microcarriers which can be highly variable in size and shape, unlike the typically spherical shape of PSC cell-only aggregates. Free-floating cells could then attach in the spaces between microcarriers, creating 3D cell formation instead of the desired 2D monolayer and thus preventing nutrients from reaching cells in the interior of those spaces. The aforementioned method of periodic dissociation can also be applied to cell-and-microcarrier clumps as well, in order to separate them into much smaller clumps of acceptable size, back into individual microcarriers with cells still attached to their surfaces, or even to completely remove cells from microcarriers. Considering the typical size of microcarriers (150-250 microns) and known desire to prevent more than 3-4 microcarriers clumping together, dissociation can be initiated when cell-and-microcarrier clumps reach more than 600 microns in diameter. Cells will proliferate on the surface of a microcarrier until there is no more space available, so in the case where dissociation will also remove cells from the surface of microcarriers, the cell density in a bioreactor post-dissociation will be greater than the initial seeding density. Therefore, new microcarriers could be added after the dissociation and medium exchange steps in order to provide more surfaces for the increased number of free-floating cells to attach and grow, leading to higher cell attachment efficiency and total cell yield in a single bioreactor.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

It is claimed:

1. A method of controlling the growth of cells and aggregates thereof, comprising:
a. seeding a bioreactor with anchorage-dependent cells;
b. operating the bioreactor for an initial period of time such that the cells form cell aggregates that will continue to grow in size and thus also increase the total number of cells in the bioreactor;
c. dissociating the cell aggregates within the same bioreactor; and
d. repeating the steps of operating and dissociating within the same bioreactor until a desired number of cells has been reached or the capacity of the bioreactor is fully utilized.

2. The method of claim 1, wherein the cells are selected from the group consisting of pluripotent stem cells (PSCs), mesenchymal stem cells (MSCs), human primary cells, or any other anchorage-dependent cells that require aggregate formation for growth.

3. The method of claim 1 where dissociating the cell aggregates can be accomplished using either a dissociation medium containing proteolytic enzymes or chemical reagents, mechanical agitation, or a combination of these methods.

4. The method of claim 1 where the timing of dissociation is dictated by cell aggregates reaching a predetermined threshold size, or range of sizes.

5. A method of controlling the growth of cells and aggregates thereof, comprising:
a. seeding a bioreactor with anchorage-dependent cells as suspended single cells, along with microcarriers;
b. operating the bioreactor for an initial period of time such that the cells first attach to the surface of microcarriers before cell-to-cell attachment leads to formation of aggregates comprised of both cells and microcarriers;
c. dissociating the aggregates comprised of cells and microcarriers within the same bioreactor; and
d. repeating the steps of operating and dissociating within the same bioreactor until a desired number of cells has been reached or the capacity of the bioreactor is fully utilized.

6. The method of claim 5, wherein the cells are selected from the group consisting of pluripotent stem cells (PSCs), mesenchymal stem cells (MSCs), human primary cells, or any other anchorage-dependent cells.

7. The method of claim 1 where dissociating the cell-and-microcarrier aggregates can be accomplished using either a dissociation medium containing proteolytic enzymes or chemical reagents, mechanical agitation, or a combination of these methods;

8. The method of claim 1c, where the timing of dissociation is dictated by the cell-and-microcarrier aggregates reaching a predetermined threshold size, or range of sizes.
